(19) United States
(12) Patent Application Publication
(10) Pub. No.: US 2007/0106078 A1
(43) Pub. Date: May 10, 2007

(54) Process for the preparation of fexofenadine

(76) Inventors: Mukesh Kumar Sharma, Haryana (IN); Chandra Has Khanduri, Haryana (IN); Naresh Kumar, Chandigarh (IN); Yatendra Kumar, Haryana (IN)

Correspondence Address: RANBAXY INC., 600 COLLEGE ROAD EAST, SUITE 2100, PRINCETON, NJ, 08540, US

(21) Appl. No.: 10/543,517

(22) PCT Filed: Jan. 30, 2004
(86) PCT No.: PCT/IB04/00233

§ 371 Date: Aug. 24, 2006

(30) Foreign Application Priority Data
Jan. 31, 2003 (IN) 79/DEL/2003

(51) Int. Cl. C07D 211/34

(52) U.S. Cl. 546/239

(57) ABSTRACT

The invention relates to highly pure fexofenadine and a process for preparing highly pure fexofenadine. The invention also relates to pharmaceutical compositions that include the highly pure fexofenadine and use of said compositions for treating a patient for allergic reactions.

### FIELD OF THE INVENTION

The field of the invention relates to highly pure fexofenadine and a process for preparing highly pure fexofenadine of structural Formula I. The invention also relates to pharmaceutical compositions that include the highly pure fexofenadine and use of said compositions for treating a patient for allergic reactions.

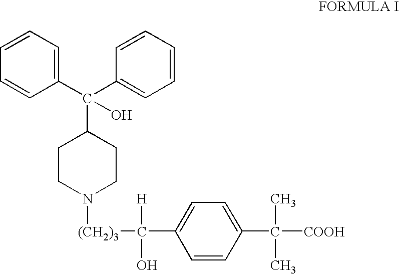

### BACKGROUND OF THE INVENTION

Chemically, fexofenadine is 4[1-hydroxy-4-[4-(hydroxybiphenylmethyl)-1-piperidinyl]butyl]-α,α-dimethylbenzene acetic acid, of structural Formula I, and is known from U.S. Pat. No. 4,254,129. It is one of the most widely used antihistamines for the treatment of allergic rhinitis, asthma and other allergic disorders.

In general, the synthetic approach reported in the literature for the preparation of fexofenadine involves the reduction of the ketone group of a carboxylate derivative, 4-[4-[4-(hydroxybiphenylmethyl)-1-piperidinyl]-1-oxobutyl]-α,α-dimethylbenzene acetate of structural Formula II, 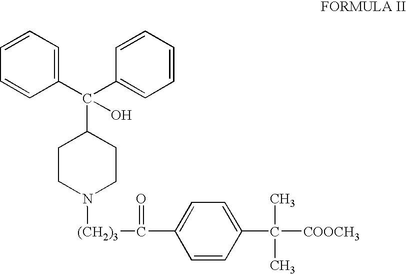
to get the corresponding hydroxyl derivative of structural Formula III, 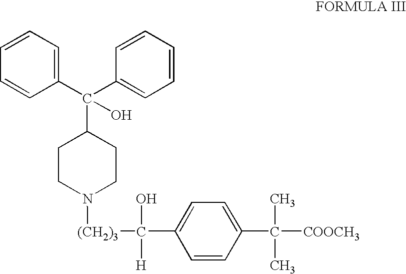
followed by hydrolysis with a base, for example alkali metal hydroxides to get a carboxylic acid derivative, fexofenadine.

There are significant drawbacks to this approach as the reduction of the ketone group to the corresponding hydroxyl derivative of structural Formula III results in the formation of many impurities, of which the following impurities are difficult to remove:

a. 4-[4-[4-(hydroxybiphenylmethyl)-1-piperidinyl]-1-oxobutyl]-α,α-dimethylbenzeneacetic acid, the impurity referred to as keto analog of fexofenadine, of structural Formula IV, and

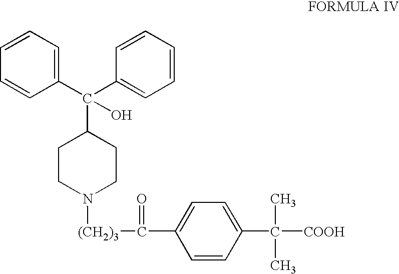

b. Meta-isomer of fexofenadine of Formula V.

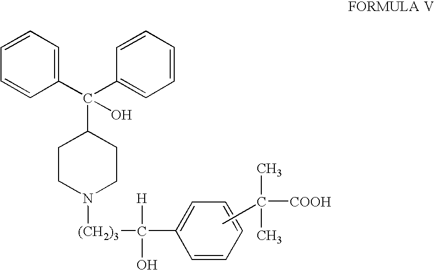

These impurities are further carried into the fexofenadine.

The prior art approach is not suitable from commercial point of view because the desired para-isomer of fexofenadine is not obtained in high purity and requires purification by tedious and cumbersome purification processes. The generation of significant quantity of unwanted meta-isomer and lower yields makes the process uneconomical.

The inventors have observed that during the reduction of methyl 4-[4-[4-(hydroxybiphenyl methyl)-1-piperidinyl]-1-oxobutyl]-α,α-dimethylphenyl acetate of structural Formula II, the product precipitates out as soon as about 80-90% conversion is achieved. Once the product is precipitated, it does not allow the reaction to go to completion and the unreacted starting material leads to the formation of impurities in the final product. To achieve a high efficiency of the reaction for industrial synthesis of fexofenadine, it is necessary to minimize the formation of the impurities and improve the yields.

Thus, the present invention provides a process for the preparation of highly pure fexofenadine which does not require any further purification.

### SUMMARY OF THE INVENTION

In one general aspect there is provided a highly pure fexofenadine or a salt thereof.

In another general aspect there is provided substantially pure fexofenadine or a salt thereof having its keto analog less than 0.05%.

In another general aspect there is provided highly pure fexofenadine or a salt thereof having its keto analog and meta-isomer, each less than 0.05%.

In another general aspect there is provided a pharmaceutical composition that includes a therapeutically effective amount of highly pure fexofenadine or a salt thereof having its keto analog and meta-isomer, each less than 0.05%; and one or more pharmaceutically acceptable carriers, excipients or diluents.

In another general aspect there is provided a process for the preparation of substantially pure fexofenadine or a salt thereof. The process includes reducing methyl 4-[4-[4-(hydroxybiphenylmethyl)-1-piperidinyl]-1-oxobutyl]-α,α-dimethyl phenyl acetate of structural Formula II, with a reducing agent to produce a reduced product methyl 4-[4-[4-(hydroxybiphenyhnethyl)-1-piperidinyl]-1-hydroxybutyl]-α,α-dimethyl phenyl acetate of structural Formula III; hydrolyzing the reduced product of structural Formula III in the presence of a base and a reducing agent; and isolating the substantially pure fexofenadine or a salt thereof.

In general, fexofenadine prepared by any of the methods known in the literature may be purified to get substantially pure or highly pure fexofenadine or a salt thereof using the process of the present invention.

In another general aspect there is provided a process for the preparation of substantially pure fexofenadine or a salt thereof. The process includes treating fexofenadine containing corresponding keto analog as an impurity with a base; adding reducing agent; and isolating the substantially pure fexofenadine having keto analog less than 0.05%.

In another general aspect there is provided a process for the preparation of highly pure fexofenadine or a salt thereof The process includes treating fexofenadine containing corresponding meta-isomer as an impurity with a base; adding acid; and isolating the highly pure fexofenadine having keto analog and meta-isomer, each less than 0.05%.

The process may include drying of the product obtained.

The base may include one or more of alkali metal hydroxide, amide, alkoxide, alkali metal, or mixtures thereof. In particular, the base is alkali metal hydroxide. The alkali metal hydroxide may be lithium hydroxide, sodium hydroxide, or potassium hydroxide. In particular, the hydroxide is sodium hydroxide.

The reducing agent may be sodium borohydride, potassium borohydride, tetralkyl ammonium borohydride, or zinc borohydride. In particular, the reducing agent is sodium borohydride.

The details of one or more embodiments of the inventions are set forth in the description below. Other features, objects and advantages of the inventions will be apparent from the description and claims.

### DETAILED DESCRIPTION OF THE INVENTION

The inventors have developed an efficient process for the preparation of substantially pure fexofenadine or a salt thereof, by reducing methyl 4-[4-[4-(hydroxybiphenylmethyl)-1-piperidinyl]-l-oxobutyl]-α,α-dimethyl phenyl acetate of structural Formula II, with a reducing agent to produce a reduced product methyl 4-[4-[4-(hydroxybiphenylmethyl)-1-piperidinyl]-1-hydroxybutyl]-α,α-dimethyl phenyl acetate of structural Formula III; hydrolyzing the reduced product of structural Formula III in the presence of a base and a reducing agent; and isolating the substantially pure fexofenadine or a salt thereof.

In general, the methyl 4-[4-[4-(hydroxybiphenylmethyl)-1-piperidinyl]-1-oxobutyl]-α, α-dimethyl phenyl acetate may be treated with a reducing agent in the presence of a solvent, and the reducing agent may be added in small lots.

The reducing agent includes any reducing agent which is capable of carrying out the reduction of the keto group, including, for example, sodium borohydride, potassium borohydride, tetralkyl ammonium borohydride, or zinc borohydride. In particular, the reducing agent is sodium borohydride.

In general, after the reduction is complete, the reaction mass is acidified and the product is filtered. The reaction mass may be acidified with any acid, including, for example, acetic acid.

In general, a solution of a base may be prepared by dissolving in water and treating the reduced product methyl 4[4-[4-(hydroxybiphenylmethyl)-1-piperidinyl]-1-hydroxybutyl]-α, α-dimethyl phenyl acetate with said solution. Alternatively, such a solution may be prepared in any solvent in which the base is soluble, including, for example, lower alkanols.

The lower alkanol may include one or more of primary, secondary and tertiary alcohol having from one to six carbon atoms. The lower alkanol may include one or more of methanol, ethanol, denatured spirit, n-propanol, isopropanol, isobutanol, n-butanol and t-butanol. In particular, the lower alkanol may include methanol and ethanol. Mixtures of all of these solvents are also contemplated.

The base includes alkali metal hydroxides, amides, alkoxides and alkali metals. The alkali metal hydroxides include any hydroxide, including, for example, lithium hydroxide, sodium hydroxide, and potassium hydroxide.

The product may be isolated from the solution by a technique which includes, for example, filtration, filtration under vacuum, decantation, and centrifugation.

The product may be further or additionally dried to achieve the desired moisture values. For example, the product may be further or additionally dried in a tray drier, dried under vacuum and/or in a Fluid Bed Drier.

The inventors have also developed a process for the preparation of substantially pure fexofenadine or a salt thereof, by treating the fexofenadine containing corresponding keto analog as an impurity, with a base; adding reducing agent; and isolating the substantially pure fexofenadine or a salt thereof having keto analog less than 0.05% as determined by Reverse Phase—HPLC.

The inventors have also developed a process for the preparation of highly pure fexofenadine or a salt thereof, by treating fexofenadine containing corresponding meta-isomer as an impurity, with a base; adding acid; and isolating the highly pure fexofenadine or a salt thereof having keto analog and meta-isomer, each less than 0.05% as determined by Reverse Phase—HPLC.

The highly pure fexofenadine or a salt thereof thus obtained contains less than 0.1% of total impurities as determined by Reverse Phase—HPLC.

Methods known in the art may be used with the process of this invention to enhance any aspect of this invention. The slurry containing the product may be cooled prior to isolation to obtain better yields of the fexofenadine and the product may be washed with a suitable solvent.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLE 1

Preparation of Substantially pure fexofenadine

### Step A: Preparation of Methyl 4-[4-[4-(hydroxybiphenylmethyl)-1-piperidinyl]-1-hydroxybutyl]-α, α-dimethyl phenyl acetate

Methyl 4-[4-[4(hydroxydiphenylmethyl)-1-piperidinyl]-1-oxobutyl]-2,2-dimethylphenylacetate (20 g) was added to methanol (60 ml), at 25-35° C. followed by the addition of solid sodium borohydride (0.81 g) in small portions. The reaction mixture was further stirred at 25-35° C. for 2-3 hours and monitored by HPLC. The reaction was quenched with acetic acid and cooled to 0-5° C. The solid was filtered and washed with cold methanol, dried to get methyl 4-[4-[4-(hydroxybiphenylmethyl)-1-piperidinyl]-1-hydroxybutyl]-α, α-dimethyl phenyl acetate (18-18.5 g).

### Step B: Preparation of Substantially Pure fexofenadine

Methyl 4-[4-[4-(hydroxybiphenylmethyl)-1-piperidinyl]-1-hydroxybutyl]-α,α-dimethylphenyl acetate (200 g) obtained in Step A was added to a mixture of ethanol (95%, 600 ml) and sodium hydroxide (23.2 g), and heated to reflux for about 3-4 hours. The reaction mixture was cooled to 50° C. and a solution of sodium borohydride (0.8 g) and sodium hydroxide (0.8 g) in water (10 ml) was added. The reaction mixture was again heated to reflux for about 1 hour and cooled to 8-10° C.; the product was filtered and washed with water and ethanol (95%). The material was dried to give 162 g of substantially pure product having keto analog less than 0.05%.

### EXAMPLE 2

Preparation of Highly Pure fexofenadine

### Step A: Preparation of Methyl 4-[4-[4-(hydroxybiphenylmethyl)-1-piperidinyl]-1-hydroxybutyl]-α, α-dimethyl phenyl acetate

Methyl 4[4-[4-(hydroxydiphenylmethyl)-1-piperidinyl]-1-oxobutyl]-2,2-dimethylphenylacetate (20 g) was added to methanol (60 ml), at 25-35° C. followed by the addition of solid sodium borohydride (0.81 g) in small portions. The reaction mixture was further stirred at 25-35° C. for 2-3 hours and monitored by HPLC. The reaction was quenched with acetic acid and cooled to 0-5° C. The solid was filtered and washed with cold methanol, dried to get methyl 4-[4-[4-(hydroxybiphenylmethyl)-1-piperidinyl]-1-hydroxybutyl]-α, α-dimethyl phenyl acetate (18-18.5 g).

### Step B: Preparation of Highly Pure fexofenadine

Methyl 4-[4-[4-(hydroxybiphenyhnethyl)-1-piperidinyl]-1-hydroxybutyl]-α,α-dimethylphenyl acetate (200 g) obtained in Step A was added to a mixture of ethanol (95%, 600 ml) and sodium hydroxide (23.2 g), and heated to reflux for about 3-4 hours.

The reaction mixture was cooled to 50° C. and a solution of sodium borohydride (0.8 g) and sodium hydroxide (0.8 g) in water (10 ml) was added. The reaction mixture was again heated to reflux for about 1 hour and cooled to 8-10° C.; the product was filtered and washed with water and ethanol (95%).

The wet product was suspended in ethanol (95%, 800 ml) and dissolved by adding a solution of sodium hydroxide (12.9 g) in water (12.9 ml). The solution was heated to 50° C. and the pH was adjusted to 6.7- 6.8 by adding 1:1 dilute hydrochloric acid. The product was isolated by cooling and filtration. The product was further dried to yield highly pure fexofenadine having keto analog and meta-isomer, each less than 0.05%.

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. A process for the preparation of substantially pure fexofenadine of structural Formula I, or a salt thereof, 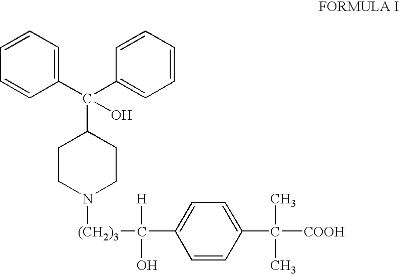
the process comprising reducing methyl 4-[4-[4-(hydroxybiphenylmethyl)-1-piperidinyl]-1-oxobutyl]-α,α-dimethylphenyl acetate of structural Formula II, 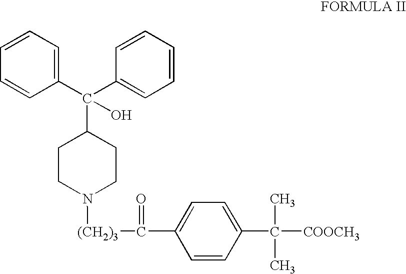
with a reducing agent to produce a reduced product methyl 4-[4-[4-(hydroxybiphenylmethyl)-1-piperidinyl]-1-hydroxybutyl]-α,α-dimethyl phenyl acetate of structural Formula III; 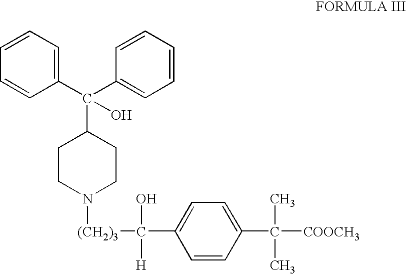
hydrolyzing the compound of Formula III in the presence of a base and a reducing agent; and isolating the substantially pure fexofenadine or a salt thereof.

2. The process of claim 1, wherein the base comprises one or more of alkali metal hydroxide, amide, alkoxide, alkali metal, or mixtures thereof.

3. The process of claim 2, wherein the alkali metal hydroxide comprises one or more of lithium hydroxide, sodium hydroxide, and potassium hydroxide.

4. The process of claim 1, wherein the reducing agent comprises one or more of sodium borohydride, potassium borohydride, tetralkyl ammonium borohydride, and zinc borohydride.

5. The process of claim 4, wherein the reducing agent is sodium borohydride.

6. The process of claim 1, wherein isolating the substantially pure fexofenadine comprises one or more of filtration, filtration under vacuum, decantation, and centrifugation.

7. The process of claim 1, further comprising additional drying of the product.

8. The process of claim 1, wherein the reduction is carried out in the presence of one or more of solvents.

9. The process of claim 8, wherein the solvent comprises one or more lower alkanols.

10. The process of claim 9, wherein the lower alkanol comprises one or more of primary, secondary and tertiary alcohols having from one to six carbon atoms.

11. The process of claim 10, wherein the lower alkanol comprises one or more of methanol, ethanol, denatured spirit, n-propanol, isopropanol, n-butanol, isobutanol, and t-butanol.

12. The process of claim 11, wherein the lower alkanol comprises one or more of methanol and ethanol.

13. The process of claim 1, wherein the reduced product is isolated.

14. The process of claim 13, wherein isolating the reduced product comprises one or more of filtration, filtration under vacuum, decantation, and centrifugation.

15. The process of claim 13, further comprising additional drying of the product.

16. A process for the preparation of substantially pure fexofenadine of structural formula I, or a salt thereof, 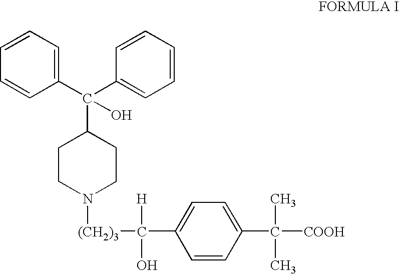
the process comprising treating fexofenadine in the presence of a base and a reducing agent; and isolating the substantially pure fexofenadine or a salt thereof.

17. The process of claim 16, wherein the base comprises one or more of alkali metal hydroxide, amide, alkoxide, alkali metal, or mixtures thereof.

18. The process of claim 17, wherein the alkali metal hydroxide comprises one or more of lithium hydroxide, sodium hydroxide, and potassium hydroxide.

19. The process of claim 16, wherein the reducing agent comprises one or more of sodium borohydride, potassium borohydride, tetralkyl ammonium borohydride, and zinc borohydride.

20. The process of claim 19, wherein the reducing agent is sodium borohydride

21. The process of claim 16, wherein isolating the substantially pure fexofenadine comprises one or more of filtration, filtration under vacuum, decantation, and centrifugation.

22. The process of claim 16, further comprising additional drying of the product.

23. A process for the preparation of highly pure fexofenadine of structural Formula I, or a salt thereof, 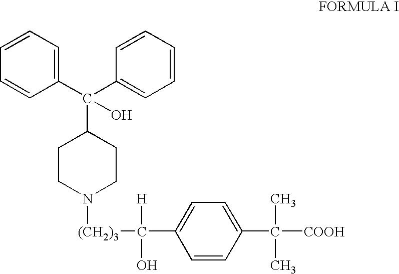
having less than 0.05% of meta-isomer impurity of Formula V, 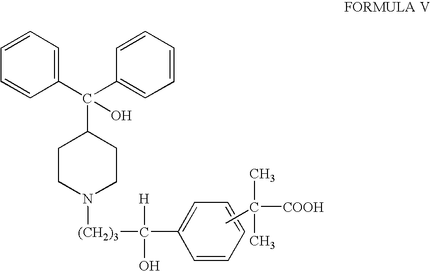
the process comprising treating fexofenadine with a base; adding acid; and isolating the highly pure fexofenadine or a salt thereof.

24. The process of claim 23, wherein the base comprises one or more of alkali metal hydroxide, amide, alkoxide, alkali metal, or mixtures thereof.

25. The process of claim 24, wherein the alkali metal hydroxide comprises one or more of lithium hydroxide, sodium hydroxide, and potassium hydroxide.

26. The process of claim 23, wherein isolating the highly pure fexofenadine comprises one or more of filtration, filtration under vacuum, decantation, and centrifugation.

27. The process of claim 23, further comprising additional drying of the product.

28. The process of claim 23, wherein the fexofenadine is treated with a base in the presence of one or more of solvents.

29. The process of claim 28, wherein the solvent comprises one or more lower alkanols.

30. The process of claim 29, wherein the lower alkanol comprises one or more of primary, secondary and tertiary alcohols having from one to six carbon atoms.

31. The process of claim 30, wherein the lower alkanol comprises one or more of methanol, ethanol, denatured spirit, n-propanol, isopropanol, n-butanol, isobutanol, and t-butanol.

32. The process of claim 31, wherein the lower alkanol comprises one or more of methanol and ethanol.

33. A method of treating allergic reactions in a patient in need thereof, the method comprising providing a dosage form to said patient that includes substantially pure fexofenadine or a salt thereof prepared by the process of claims 1 or 16.

34. A method of treating allergic reactions in a patient in need thereof, the method comprising providing a dosage form to said patient that includes highly pure fexofenadine or a salt thereof prepared by the process of claim 23.

35. (canceled)

36. Highly pure fexofenadine or a salt thereof having keto analog and meta-isomer, each being present at an amount less than 0.05%.

37. Substantially pure fexofenadine or a salt thereof having keto analog being present at an amount less than 0.05%.

38. A pharmaceutical composition comprising a therapeutically effective amount of highly pure fexofenadine or a salt thereof prepared by the process of claim 23; and one or more pharmaceutically acceptable carriers, excipients or diluents.

39. (canceled)

40. A pharmaceutical composition comprising a therapeutically effective amount of substantially pure fexofenadine or a salt thereof prepared by the process of claim 1 or 16; and one or more pharmaceutically acceptable carriers, excipients or diluents.

41. (canceled)
